(19) United States
(12) Patent Application Publication
(10) Pub. No.: US 2013/0190677 A1
(43) Pub. Date: Jul. 25, 2013

(54) PHOTO-STIMULATION METHOD AND KIT WITH AGONIST AGENT

(71) Applicants: Ming-Chieh TU, Keelung City (TW); Yi-Wei HSIAO, New Taipei City (TW); Chung-Pei LEE, Taipei City (TW); Jung-Chien CHANG, New Taipei City (TW); Xin-Fan HUANG, Taipei City (TW); Yu-Chia TSAO, Taipei City (TW)

(72) Inventors: Ming-Chieh TU, Keelung City (TW); Yi-Wei HSIAO, New Taipei City (TW); Chung-Pei LEE, Taipei City (TW); Jung-Chien CHANG, New Taipei City (TW); Xin-Fan HUANG, Taipei City (TW); Yu-Chia TSAO, Taipei City (TW)

(73) Assignee: Forward Electronics Co., Ltd., Taipei City (TW)

(21) Appl. No.: 13/470,590

(22) Filed: May 14, 2012

(30) Foreign Application Priority Data
Jan. 19, 2012 (TW) 101102140

(51) Int. Cl. A61M 37/00

(52) U.S. Cl. 604/20

(57) ABSTRACT

Disclosed is a photo-stimulation method employing an agonist agent, and a kit for introducing same. The method includes the following steps: providing a light-emitting diode (LED) illuminant which is a yellow, red, green, blue LED or a mixture of two or more kinds thereof, and an agonist agent which contains 0.5% to 2% calcium ion; and adding the agonist agent to a subject and illuminating the subject by the LED illuminant to promote collagen synthesis, to suppress microbial growth, or to inhibit melanin synthesis, wherein the yellow LED is in an illuminance range from 1,000 to 3,500 lux, the red LED is in an illuminance range from 6,000 to 9,500 lux, the green LED is in an illuminance range from 1000 to 5000 lux, and the blue LED is in an illuminance range from 3,000 to 7,000 lux.

### CROSS REFERENCE TO RELATED APPLICATION

This application claims the benefits of the Taiwan Patent Application Serial Number 101102140, filed on Jan. 19, 2012, the subject matter of which is incorporated herein by reference.

### BACKGROUND OF THE INVENTION

1. Field of the Invention

The present invention relates to a photo-stimulation method and a kit, more particularly, to a photo-stimulation method and a kit with agonist agent. The present invention can promote collagen synthesis, suppress microbial growth, or inhibit melanin synthesis.

2. Description of Related Art

After dermatological diagnosis, drugs are generally used to treat patients' skin conditions, such as acne. However, drug therapy frequently incurs side effects and long-term drug administration also results in metabolic loads on patients. Notably, such therapy does not bring desirable efficacy of treatment and treated patients often have a relapse of skin conditions. Hence, patients' skin conditions can not be efficiently eradicated.

In recent years, medical cosmetology has been greatly developed. Some research reported blue light, which is in a wavelength range from 400 to 475 nm, could be applied for acne treatment. Since photosensitive coproporphyrin in Propionibacterium acnes (P. acnes) or tissue cells generate toxic mono-oxygen and free radicals when reacted with blue light, microbes and some cells of the sebaceous gland tissue are consequently killed. Hence, the blue light reduces the inflammation of acne.

In addition, red light with a wavelength of 600-750 nm, yellow light with a wavelength of 550-600 nm, and green light with a wavelength of 500-570 nm can stimulate fibroblasts in the dermis to induce synthesis of collagen and to prevent skin aging.

At present, in order to achieve the above mentioned effects, laser or intense pulsed light is often applied in the industry of medical cosmetology. However, owing to high energy and intensity of the light, it is easy for the aforesaid light to cause injury to cells. General light sources or light-emitting diodes (LEDs) have been recently developed to replace the high-intensity light above. Due to the relatively low energy of light emitted from LEDs, appropriate illuminance of the light needs to be found to achieve the aforesaid effects. Too low illuminance of light does not induce good treatment, and, conversely, too high illuminance of light injures cells and has to be generated by large LED devices. Accordingly, it is difficult to create a compact and portable LED device for phototherapy.

Therefore, it is desirable to provide a photo-stimulation method and a kit with an agonist agent that promotes collagen synthesis and increases the suppression of microbial growth or the inhibition of melanin synthesis so that labor, power, and time costs can be economized and the treated patients' skin conditions can be improved.

### SUMMARY OF THE INVENTION

The major object of the present invention is to provide a photo-stimulation method with an agonist agent, which can promote collagen synthesis and increase the suppression of microbial growth or inhibition of melanin synthesis.

To achieve the object, the photo-stimulation method of the present invention comprises the following steps: providing a light-emitting diode (LED) illuminant which is a yellow LED, a red LED, a green LED, a blue LED or a combination thereof, and an agonist agent which contains 0.5% to 2% calcium ion; and adding the agonist agent to a subject and illuminating the subject by the LED illuminant to promote collagen synthesis, to suppress microbial growth, or to inhibit melanin synthesis, wherein the yellow LED is in an illuminance range from 1,000 to 3,500 lux (lx), the red LED is in an illuminance range from 6,000 to 9,500 lux, the green LED is in an illuminance range from 1,000 to 5,000 lux, and the blue LED is in an illuminance range from 3,000 to 7,000 lux.

In the photo-stimulation method of the present invention, the agonist agent contains 0.5% to 2% calcium ion, preferably containing 1% to 2% calcium ion, and more preferably, containing 1.5% to 2% calcium ion. The agonist agent cannot function efficiently while the concentration of calcium ion is too low or too high. Besides, the agonist agent can contain any constituents that have been used as a buffer or carrier in the art, and the constituents will not influence cell morphology, cell proliferation, and cell metabolism. Moreover, the form of the agonist agent is not limited; it can be powder, liquid, gel, or solid. Furthermore, the way of adding the agonist agent is also not limited, for the agonist agent only has to come in contact with the subject.

Due to the aforesaid reason, after the agonist agent is added to the subject, the subject is illuminated by the illuminant and then the agonist agent is removed. If the subject will be illuminated for a second or more times, the more agonist agent can be added in intervals if necessary between each illumination period. However, the used agonist agent has to be removed and replaced with fresh agonist prior to the next illuminating procedure.

Accordingly, the photo-stimulation method with agonist agent of the present invention provides an LED illuminant stimulation method with agonist agent. In this embodiment, the subject is a fibroblast, a macrophage, or, preferably, a combination thereof.

In the photo-stimulation method with agonist reagent, the LED illuminant is preferably, but not limited to, a yellow, red, or green LED. Furthermore, the wavelength of the yellow LED could range from 550 to 600 nm, the wavelength of the red LED could range from 600 to 750 nm, and the wavelength of the green LED could range from 500 to 570 nm. The above mentioned yellow LED is in an illuminance range from 1,000 to 3,500 lux, preferably from 2,000 to 2,500 lux; the red LED is in an illuminance range from 6,000 to 9,500 lux, preferably from 7,500 to 8,500 lux; and the green LED is in an illuminance range from 1,000 to 5,000 lux, preferably from 2,000 to 3,500 lux.

In addition, the illuminating time of the LED illuminant is not limited, as long as the aforementioned efficiency is achieved without resulting in any injury to the subject. The illuminating time of the LED illuminant could be modulated judging from the decided illuminance of light emitted from the LEDs. When the illuminance is high, it can perform the same effect with a shorter illuminating time. On the contrary, when the illuminance is low, it needs a longer illuminating time to perform the same effect. In the preferred embodiment of the present invention, the illuminating time of the LED illuminant is preferably 5 to 30 min. Moreover, the number of treatment times for illuminating the subject is twice or more, but not limited to, and the intervals between the illuminating times could be 1 to 36 hours, preferably be 12 to 24 hours, and more preferably is 24 hours.

Illuminating by the LED illuminant can stimulate the fibroblasts, and increase the amount of collagen from synthesis because cytokine released from the macrophage can further stimulate the fibroblasts. Additionally, parts of the LED illuminant as red LED, or green LED can directly stimulate the DNA synthesis of fibroblasts, or increase the secretion of fibroblast growth factor (FGF) from fibroblasts. Since the above three reasons show the positive correlation to the concentration of calcium ion in the fibroblasts, the addition of the agonist agent while illuminating the fibroblasts by the LED illuminant can effectively promote collagen synthesis and does not result in injury to the fibroblasts. Furthermore, if the subject is a cell in vitro, it can achieve efficiency by implanting the cell back to the organism after completing the aforementioned procedure. Accordingly, the subject of the present invention is an object illuminated by the illuminant.

According to another preferred embodiment, the subject is P. acnes, a melanocyte, or, preferably, a combination thereof A blue LED is the preferred LED illuminant, which has a wavelength ranging from 400 to 475 nm, but not limited to. Moreover, the blue LED is in an illuminance range from 3,000 to 7,000 lux, preferably from 5,000 to 5,800 lux. The illuminating time of the LED illuminant is not limited, but preferably 15 to 90 min; and the number of times of illuminating the subject is not limited, either.

Illuminating P. acnes by the blue LED illuminant and adding the agonist agent can inhibit the microbial growth effectively. In the preferred embodiment of the present invention, illuminating P. acnes by the blue LED illuminant in conjunction with the agonist agent, for 60 minutes can completely (100%)inhibit P. acnes.

In order for melanocyte to synthesize melanin, the presence of tyrosinase is required. While the blue illuminant can inhibit melanin synthesis effectively, calcium ions can inhibit the activity of tyrosinase directly. Hence, when illuminating the melanocyte by the blue LED illuminant, adding the agonist agent can promote the inhibition of melanin synthesis and precipitation effectively, and does not result in injury to the melanocyte.

If the subject is skin, for acne pocks or P. acnes on the skin surface, including the agonist agent when illuminating by the illuminant can regulate division and differentiation of skin cells, decrease sebum secretion, and control the proliferation of epidermal cells in the vessel area of the follicle. Consequently, the present invention can achieve the killing effect for P. acnes and the treatment effect of acne such as reducing the inflammation. On the other hand, for melanocyte on the skin surface, including the agonist agent when illuminating by the illuminant can promote the inhibition of melanin synthesis, avoid increasing skin chrominance and then achieve a skin whitening effect.

Another object of the present invention is to provide a photo-stimulation kit, comprising: a photo-stimulation device and an agonist agent. In the device, LEDs are set to emit red, yellow, or green light in a specific range of illuminance so as to stimulate collagen synthesis of the fibroblasts and to promote blood circulation as well as accelerate the removal of dead cells. Alternatively, in the photo-stimulation device, LEDs which emit blue light in a specific range of illuminance can inhibit or kill P. acnes, or decrease the melanin synthesis of melanocytes.

To use the photo-stimulation kit of the present invention, add the agonist agent to a subject before illuminating, and then use the photo-stimulation device.

In order to achieve the object, the photo-stimulation device comprises a casing, a diffuser plate, a first illuminant module, and a controller. The casing forms a deposition space and has a top surface and a lateral surface, and the top surface is provided with a light-output window; a diffuser plate covers the light-output window of the casing; a first illuminant module is deposed in the deposition space of the casing and has a first light-emitting diode (LED) located under the diffuser plate, and the first light-emitting diode is selected from a group consisting of a red LED, a yellow LED, a green LED, and a blue LED, wherein the light emitted from the yellow LED and passing through the diffuser plate has an illuminance ranging from 1,000 to 3,500 lux (lx), the light emitted from the red LED passing through the diffuser plate has an illuminance ranging from 6,000 to 9,500 lux, the light emitted from the green LED passing through the diffuser plate has an illuminance ranging from 1,000 to 5,000 lux, and the light emitted from the blue LED passing through the diffuser plate has an illuminance ranging from 3,000 to 7,000 lux; and a controller module is electrically connected with the first illuminant module and a power module.

In the photo-stimulation device of the present invention, the power module can be an external power supply or be placed in the deposition space of the casing. The power module can contain rechargeable batteries, dry batteries, or micro-batteries placed in the deposition space of the casing to supply electricity. Alternatively, if the power module is an external power supply or a rechargeable battery placed in the deposition space of the casing, the controller module can selectively further include a charge socket that provides an electrical connection between the power module and the controller module.

In the photo-stimulation device of the present invention, the controller module can selectively comprise a power switch mounted on the surface of the casing to control power output of the power module. Furthermore, the casing is preferably made of a material with low transmittance, for example, a material with high reflectivity or density for reducing light leakage of the photo-stimulation device. Also, in order to reduce light leakage of the photo-stimulation device, one skilled in the art of the present invention can increase tightness of the integral structure of the photo-stimulation device by various structural designs.

Alternatively, the lateral surface of the casing can be selectively provided with a light-output hole in the photo-stimulation device of the present invention. In this case, the photo-stimulation device can further include a light-transmission plate covering the light-output hole, and a second illuminant module deposed corresponding to the light-transmission plate and emitting light that passes through the light-transmission plate. In this state, the controller module can further include a mode switch mounted on the surface of the casing to turn on the first illuminant module or the second illuminant module, as to switch between the first illuminant module and the second illuminant module.

In the photo-stimulation device of the present invention, the diffuser plate placed on the light-output window is beneficial to uniform light emission; avoids directing light illumination on users' eyes; and increases uniformity of photo-stimulation of the device. In other words, light of LEDs that are classified into a point light passes through the diffuser plate, and then forms a surface light at the light-output window. The light-transmission plate placed on the light-output hole does not have to be a diffuser plate. If the light-transmission plate is a diffuser plate, the above-mentioned benefits can be achieved; if the light-transmission plate is not a diffuser plate, light supply can be directly transmitted from a point light source.

In the photo-stimulation device of the present invention, the first and second illuminant modules can be designed to be replaceable. In other words, red, yellow, green and blue LEDs constitute the first and second illuminant modules. If a user needs the red light illumination, the illuminant module that is constituted by red LEDs is arranged in the device. If there is a need for blue light illumination, the illuminant module that is constituted by blue LEDs is arranged in the device. Furthermore, LEDs used in the first and second illuminant modules can be designed to be replaceable. In other words, if there is a need of red light illumination, LEDs used in the first and second illuminant modules can be replaced with red LEDs.

In conventional methods, laser or intense pulsed lights with different wavelengths are applied to treat acne and stimulate fibroblasts of dermis to increase collagen synthesis. Since laser or intense pulsed light with high intensity has to be produced by large apparatus, it is difficult for general consumers to have such large apparatus. Although there has been research for LEDs which are used as a light source for acne treatment and promotion of collagen synthesis, the influence of the illuminance of LEDs on cells or bacteria is not studied in conventional research. Therefore, it is uncertain whether the conventional method can achieve the aforementioned effects with unspecified luminance of light. Conversely, in the method of the present invention, blue, green, yellow or red light of LEDs used as illuminants are adjusted in corresponding ranges of illuminance. With the addition of the agonist agent, which contains 0.5% to 2% calcium ion during the illuminating course, the present invention is sure to achieve stimulation of fibroblasts, promotion of collagen synthesis, inhibition or killing of P. acnes, and reduction or inhibition of melanin synthesis efficiently so as to treat acne pocks and carry out skin whitening or anti-aging effects.

### BRIEF DESCRIPTION OF THE DRAWINGS

FIG. 1 is a perspective view of a photo-stimulation device in Example 1 of the present invention;

FIG. 2 is a side view of a photo-stimulation device in Example 1 of the present invention;

FIG. 3 is a system block diagram of a photo-stimulation device in Example 1 of the present invention;

FIG. 4A-4C is a chart of collagen synthesis rate in Experiment 1-3 of the present invention;

FIG. 5A-5C is a chart of fibroblasts viability in Experiment 4-6 of the present invention;

FIG. 6 is a chart of melanin synthesis rate in Experiment 7 of the present invention;

FIG. 7 is a chart of melanocyte viability in Experiment 8 of the present invention; and

FIG. 8 is a chart of anti-microbial percentage in Experiment 9 of the present invention.

### DETAILED DESCRIPTION OF THE PREFERRED EMBODIMENT

Because of the specific embodiments illustrating the practice of the present invention, one skilled in the art can easily understand other advantages and efficiency of the present invention through the content disclosed herein. The present invention can also be practiced or applied by other variant embodiments. Many other possible modifications and variations of any detail in the present specification based on different outlooks and applications can be made without departing from the spirit of the invention.

The drawings of the embodiments in the present invention are all simplified charts or views, and only reveal elements relative to the present invention. The elements revealed in the drawings are not necessarily aspects of the practice, and quantity and shape thereof are optionally designed. Further, the design aspect of the elements can be more complex.

### EXAMPLE 1

### The Photo-Stimulation Device]

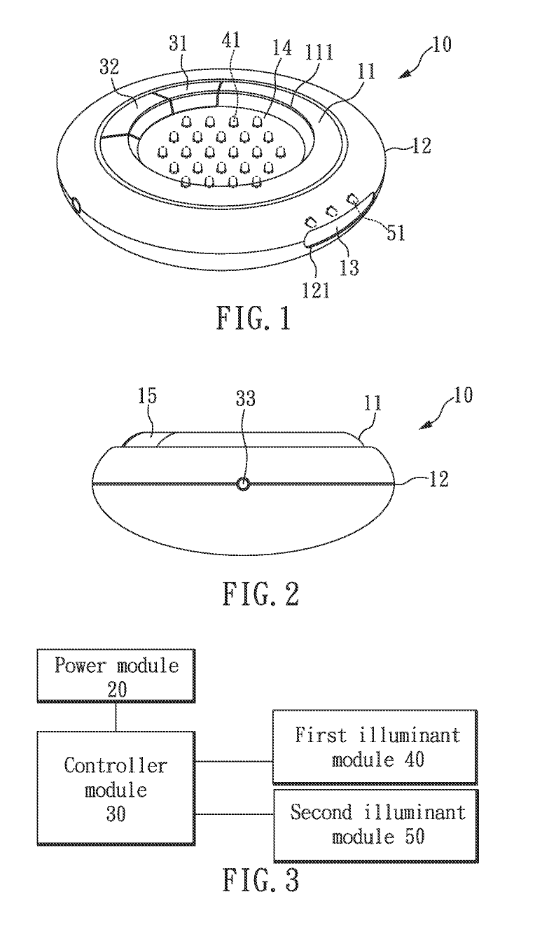

With reference to FIGS. 1 to 3, FIGS. 1 to 3 respectively show a perspective view, a side view, and a system block diagram of a photo-stimulation device of the present invention.

As shown in FIGS. 1 to 3, the photo-stimulation device of the present invention includes: a casing 10, a diffuser plate 14, a light-transmission plate 13, a first illuminant module 40, a second illuminant module 50, and a controller module 30.

The casing 10 forms a deposition space for receiving different modules. In addition, the casing 10 has a top surface 11 and a lateral surface 12. The top surface 11 is provided with a light-output window 111. The lateral surface 12 is provided with a light-output hole 121.

The light-output window 111 of the top surface 11 is covered by the diffuser plate 14, and the light-output hole 121 of the lateral surface 12 is covered by the light-transmission plate 13. The second illuminant module 50 corresponds to the light-transmission plate 13 and is placed in the deposition space of the casing 10. The second illuminant module 50 emits light passing through the light-transmission plate 13 and has one or more second LEDs 51. Herein, if the light-transmission plate 13 is used for light transmittance but not for light diffusion, the second illuminant module 50 serves as a point source of light.

The first illuminant module 40 is located in the deposition space of the casing 10 and a plurality of first LEDs 41 arranged in an array under the diffuser plate 14. The first LEDs 41 are selected from a group consisting of a red LED, a yellow LED, and a blue LED. The light passing through the diffuser plate and emitted from the yellow, red, green and blue LED has an illuminance in a range of 1,000-3,500 lux, 6,000-9,500 lux, 1,000-5,000 lux, and 3,000-7,000 lux, respectively.

The controller 30 is electrically connected with the first illuminant module 40 and a power module 20, and includes: a charge socket 33 which provides an electrical connection between the power module 20 and the controller module 30; a power switch 31 mounted on the surface of the casing 10 to control power output of the power module 20; and a mode switch 32 mounted on the surface of the casing 10 to turn on the first illuminant module 40 or the second illuminant module 50.

The power module 20 can be an external power supply or is placed in the deposition space of the casing 10. When the power module 20 is placed in the deposition space of the casing 10, the power module 20 can contain rechargeable or dry batteries or microbatteries for power supply.

Accordingly, in the photo-stimulation device, green, red or yellow LEDs that emit light in a specific range of illuminance are combined with the agonist agent and employed to stimulate fibroblasts and collagen synthesis and to promote blood circulation, as well as speed up removal of dead cells. Alternatively, blue LEDs that emit light in a specific range of illuminance are combined with the agonist agent and employed to inhibit or kill P. acnes or reduce and suppress melanin synthesis in melanocytes.

The photo-stimulation device of example 1 that can emit blue, green, red or yellow LEDs with various ranges of illuminance was used in the experiments of the present invention. Furthermore, the agonist reagent provided in the experiments was “ Calcium-T Complex” of GenePharm Biotech Corp., and the “Calcium-T Complex” mixed with ddH₂0 to form 0.25% V/V Calcium-T Complex.

[Experiment 1—Collagen Synthesis Rate with the Agonist Reagent and Red LED (Lux 8,480)]

First, human fibroblasts (2×10⁴ cells/well) were seeded with DMEM in a 48-well plate and cultured for 24 hours in an incubator at 37° C. and 5% CO2. Each well of the 48-well plate contained the cells and DMEM in a total volume of 0.5 ml.

Subsequently, all the culture media were removed, and then 500 μl PBS, which contained 0.25% agonist reagent, was added into each well. The cells were illuminated by the red LED (8,480 lux) for 5, 10, 15, and 30 minutes respectively. Then, total PBS in the well was removed and 500 μl DMEM, which contained 0.25% agonist reagent, was added into each well. The cells were incubated for another 24 hours.

Then, the procedure described in the previous paragraph is repeated. The cells were illuminated by the red LED again and cultured for 24 hours in an incubator.

The culture medium in each well was taken out and collected into tubes. 500 μl PBS was added to wash the wells then collected into another tube. An aqueous solution of acetic acid (0.5 M, 500 μl, 4° C.) was added to tubes respectively and stood for 1 hour to dissolve collagen. 1 hour later, 500 μl solution was collected by pipette into the 1.5 ml Eppendorf tubes. Then, 50 μl acid neutralizing reagent (Biocolor) and 100 μl isolation & concentration reagent (4° C., Biocolor) were added to the 1.5 ml Eppendorf tubes in sequence. The mixture stood at 4° C. overnight.

Then, the mixture was centrifuged at 12,000 rpm for 10 minutes. The supernatant was removed, and 1 ml sircol dye reagent (Biocolor) is added into the tubes respectively. The tubes were sonicated for 45 minutes at 0° C., and centrifuged at 12,000 rpm for 10 minutes. The supernatant was removed. Subsequently, 750 μl acid-salt wash reagent (Biocolor) was added into the tubes. The tubes were centrifuged at 12,000 rpm for 10 minutes.

Finally, the supernatant was removed, and 250 μl alkali reagent (Biocolor) was added into the tubes. The mixture (200 μl) of each tube was taken out and added to each well of a 96-well plate. The absorbance of the mixtures at 555 nm was measured by an ELISA Reader (SpectraMax M2). Collagen synthesis rate (%)=(Collagen synthesis after illumination/Collagen synthesis of control)×100%. In the equation, the control referred to cells that were illuminated without the agonist reagent in PBS and DMEM medium.

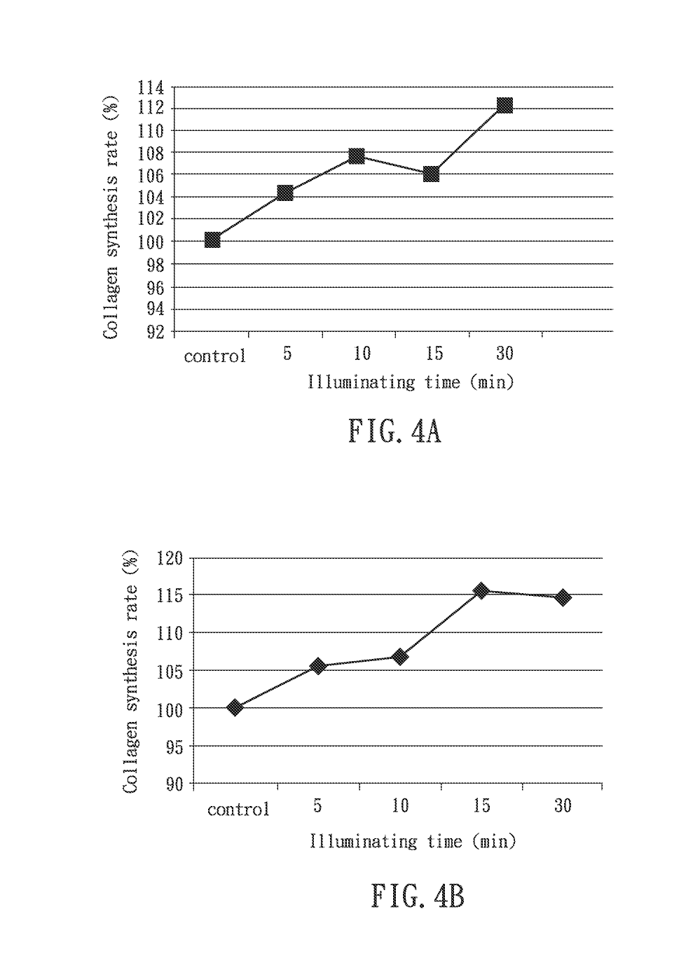

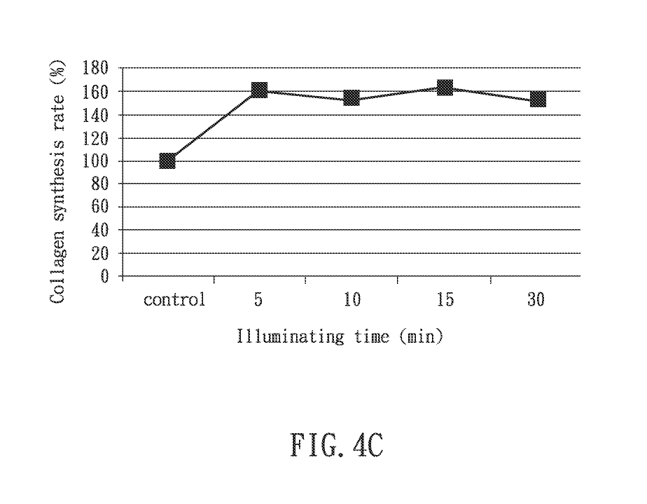

As shown in FIG. 4A, the collagen synthesis rate when illuminated with the red LED at 8,480 lux for 30 minutes is 112%. This result indicates the agonist reagent is able to promote collagen synthesis of fibroblasts.

[Experiment 2—Collagen Synthesis Rate with the Agonist Reagent and Yellow LED (Lux 2,290)]

The experimental method, procedure, and conditions were the same as described in Experiment 1, except cells were illuminated with the yellow LED (Lux 2,290) to replace the red LED (8,480 lux). The results are shown in FIG. 4B.

As shown in FIG. 4B, the collagen synthesis rate when illuminated with the yellow LED at 2,290 lux for 15 minutes is 115%. This result indicates the agonist reagent is able to promote collagen synthesis of fibroblasts.

[Experiment 3—Collagen Synthesis Rate with the Agonist Reagent and Green LED (Lux 2,700)]

The experimental method, procedure, and conditions were the same as described in Experiment 1, except cells were illuminated with the green LED (Lux 2,700) to replace the red LED (8,480 lux). The results are shown in FIG. 4C.

As shown in FIG. 4C, the collagen synthesis rate when illuminated with the green LED at 2,700 lux for 5 minutes is 160%. This result indicates the agonist reagent is able to promote collagen synthesis of fibroblasts.

[Experiment 4—Cell Viability of Human Fibroblasts with the Agonist Reagent and Red LED (Lux 8,480)]

First, human fibroblasts (2×10⁴ cells/well) were seeded with DMEM in a 48-well plate and cultured for 24 hours in an incubator. Each well of the 48-well plate contained the cells and DMEM in a total volume of 0.5 ml.

Subsequently, all the culture media were removed, and then 500 μl PBS, which contained 0.25% agonist reagent, was added into each well. The cells were illuminated by the red LED (8,480 lux) for 5, 10, 15, and 30 minutes respectively. Then, total PBS in the well was removed and 500 μl DMEM, which contained 0.25% agonist reagent, was added into each well. The cells were incubated for another 24 hours.

Then, the procedure described in the previous paragraph is repeated. The cells were illuminated by the red LED again and cultured for 24 hours in an incubator.

The culture medium in each well was replaced with 0.5 ml fresh DMEM and 0.125 ml MTT reagent (Sigma) was added into each well. Then, the cells were incubated in an incubator for 4 hours. The solution was totally removed and 0.5 ml DMSO reagent was added into the wells. After reaction was completed, the mixtures were mixed well and 0.2 ml was placed into a 96-well plate. The absorbance of the mixtures at 570 nm was measured by an ELISA Reader (SpectraMax M2). Cell viability (%)=(illuminated OD₅₇₀/control OD₅₇₀)×100%. In the equation, the definition of control was the same as described in Experiment 1.

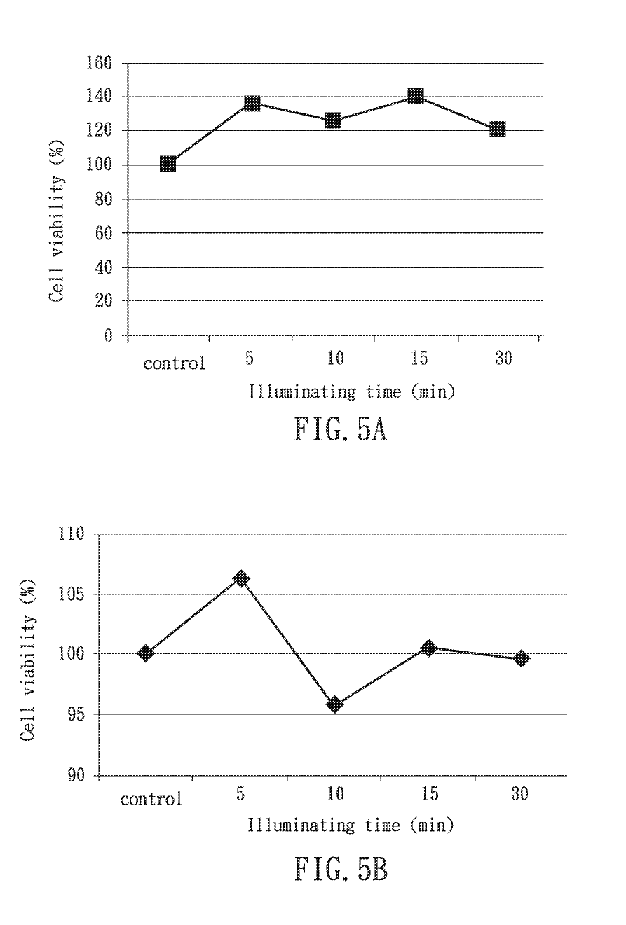

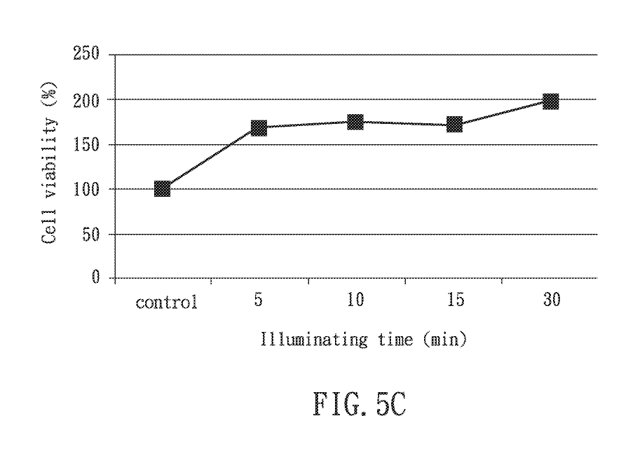

As shown in FIG. 5A, the cell viabilities when illuminated with red LED (8,480 lux) and treated with the agonist reagent during the 5-30 minutes are 120-140%, which shows a higher percentage than ±10% of human error. This result indicates the agonist reagent is able to increase the cell number of the human fibroblasts stimulated by red LED (8,480 lux).

[Experiment 5—Cell Viability of Human Fibroblasts with the Agonist Reagent and Yellow LED (Lux 2,290)]

The experimental method, procedure, and conditions were the same as described in Experiment 4, except cells were illuminated with the yellow LED (Lux 2,290) to replace the red LED (8,480 lux). The results are shown in FIG. 5B.

As shown in FIG. 5B, the cell viabilities when illuminated with the yellow LED (2,290 lux) and treated with the agonist reagent fall into the ±10% of human error during the 30 minutes. This result indicates the agonist reagent does not affect the cell viability of human fibroblasts.

[Experiment 6—Cell Viability of Human Fibroblasts with the Agonist Reagent and Green LED (Lux 2,700)]

The experimental method, procedure, and conditions were the same as described in Experiment 4, except cells were illuminated with the green LED (Lux 2,700) to replace the red LED (8,480 lux). The results are shown in FIG. 5C.

As shown in FIG. 5C, the cell viabilities when illuminated with the green LED (2,700 lux) and treated with the agonist reagent during the 5-30 minutes are 17˜200%, which shows a higher percentage than ±10% of personal error. This result indicates the agonist reagent is able to increase the cell number of the human fibroblasts stimulated by the green LED (2,700 lux).

With respect to Experiments 4 to 6, there is no cytotoxicity to human fibroblasts with the red LED (8,480 lux), the yellow LED (Lux 2,290), and the green LED (Lux 2,700) in the existence of the agonist reagent. Accordingly, the red LED (8,480 lux) and the green LED (Lux 2,700) can increase the cell numbers of human fibroblasts stimulated by LEDs more effectively. Further, evaluated with Experiment 1 to 3, after the agonist reagent treatment, human fibroblasts that are illuminated by the red LED (8,480 lux) for 30 minutes can increase cell number and collagen synthesis; human fibroblasts that are illuminated by the yellow LED (2,290 lux) for 15˜30 minutes show no sign of cell proliferation, but a significant increase in collagen synthesis; human fibroblasts that are illuminated by the green LED (2,700 lux) for 5˜30 minutes can increase cell number and collagen synthesis significantly. As a result, by adding the agonist reagent, collagen synthesis of human fibroblasts stimulated by red, yellow, and green LED can be increased effectively.

[Experiment 7—Melanin Synthesis Rate with the Agonist Reagent and Blue LED (Lux 5,330)]

First, human melanocytes (1×10⁵ cells/well) were seeded with DMEM (contained 10% FBS (Hyclone)) in a 24-well plate and cultured for 24 hours in an incubator. Each well of the 24-well plate contained the cells and DMEM in a total volume of 0.5 ml.

Subsequently, all the culture media were removed, and then 500 μl PBS, which contained 0.25% agonist reagent, was added into each well. The cells were illuminated by the blue LED (5,330 lux) for 15, 30, 45, 60, and 90 minutes respectively. Then, total PBS in the well was removed and 500 μl DMEM, which contained 0.25% agonist reagent, was added into each well. The cells were incubated for another 24 hours.

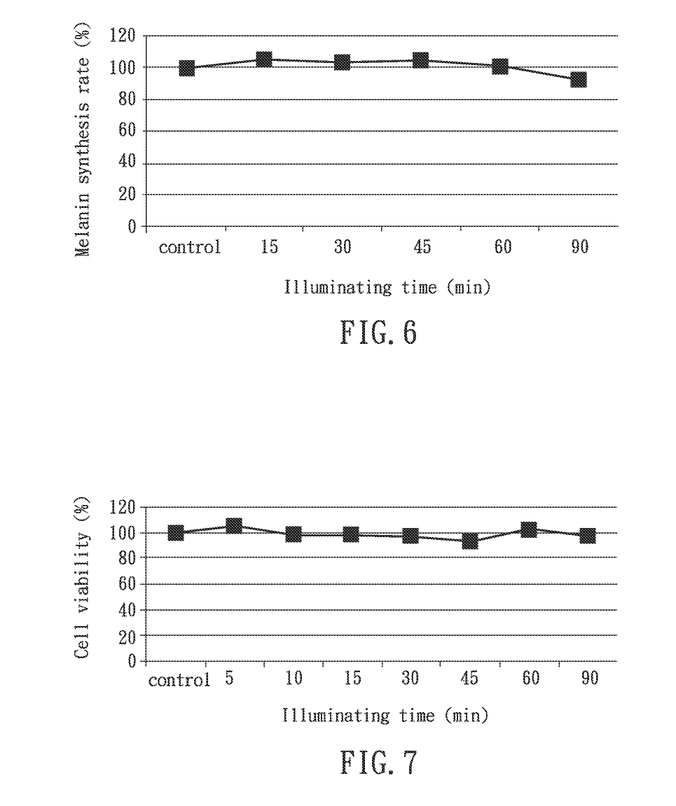

Then, the culture medium in each well was replaced with trypsin-EDTA to detach the cells from the plate. The cells were collected into a tube, and centrifuged at 1,000 rpm for 10 minutes. The supernatant was removed. Then, 200 μl NaOH aqueous solution (1 M) was added to the tube and placed in a boiling water bath for 10 minutes. Absorbance of the mixtures at 490 nm was measured by ELISA Reader (SpectraMax M2) to determine the amount of melanin. The results are shown in FIG. 6. Melanin synthesis rate (%)=(illuminated OD₄₉₀/control OD₄₉₀)×100%. In the equation, the definition of control was the same as described in Experiment 1.

As shown in FIG. 6, the melanin synthesis rate of the human melanocytes illuminated with blue LED (5,330 lux) and the agonist reagent for 90 minutes is 90%. Therefore, combining the agonist reagent with blue LED (5,330 lux) can inhibit the activity of tyrosinase and further decrease the melanin synthesis.

[Experiment 8—Cell Viability of Human Melanocytes with the Agonist Reagent and Blue LED (Lux 5,330)]

First, human melanocytes (7×10⁴ cells/well) were seeded with DMEM (contained 10% FBS (Hyclone)) in a 24-well plate and cultured for 24 hours in an incubator. Each well of the 24-well plate contained cells and DMEM in a total volume of 0.5 ml.

Subsequently, all the culture media were removed, and then 500 μl PBS, which contained 0,25% agonist reagent, was added into each well. The cells were illuminated by the blue LED (5,330 lux) for 5, 10, 15, 30, 45, 60, and 90 minutes respectively. Then, total PBS in the well was removed and 500 μl DMEM, which contained 0,25% agonist reagent, was added into each well. The cells were incubated for another 24 hours.

The culture medium in each well was replaced with 0.5 ml fresh DMEM and 0.125 ml MTT reagent (Sigma) was added into each well. Then, the cells were incubated in an incubator for 4 hours. The solution was totally removed and 0.5 ml DMSO reagent was added into the wells. After reaction was completed, the mixtures were mixed well and 0.2 ml was placed into a 96-well plate. The absorbance of the mixtures at 570 nm was measured by an ELISA Reader (SpectraMax M2). Cell viability (%)=(illuminated OD₅₇₀/control OD₅₇₀)×100%. In the equation, the definition of control was the same as described in Experiment 1.

As shown in FIG. 7, the cell viabilities illuminated with the blue LED (5,330 lux) and treated with the agonist reagent fall into the ±10% of personal error during the 90 minutes. This result indicates the agonist reagent does not affect the cell viability of human melanocytes.

With respect to Experiment 7 and 8, there is no cytotoxicity to human melanocytes with the blue LED (5,330 lux) in the existence of the agonist reagent. Accordingly, by way of inhibition of tyrosinase activity, melanin synthesis is decreased in human melanocytes.

[Experiment 9—Anti-P. acnes Percentage with the Agonist Reagent and Blue LED (Lux 5,090)]

First, a lyophilized stock culture was taken out from the refrigerator. Streaking was performed on an agar plate. Then, a single colony was picked by a sterilized loop and spread uniformly on another agar plate. After incubation for 48 hours, the bacterium was scraped from the agar plate and suspended in sterilized water. The suspension was adjusted to 0D₆₀₀=0.1 and the agonist reagent was added into the suspension. The suspension was diluted with the same volume of sterilized water so as to obtain a bacterium broth containing 10⁶ bacteria.

Subsequently, the broth (5 ml) was spread in 6 cm Petri dishes and illuminated by the blue LED (5,090 lux) photo-stimulation device of Example 1 for 5, 10, 15, 20, 30, 45, 60, and 90 minutes.

Then, the illuminated broths were ten-fold serial diluted into 10⁻³, 10⁻⁴, and 10⁻⁵. The diluted broths (0.1 ml) with respective concentrations were spread on triplicate RCM agar plates (BD biosciences) and cultured under an anaerobic condition at 37° C. for 48 hours. Then, the colony number on the plate was calculated and 30-300 colony-forming units (CFUs) found on one plate were considered as an effective number of colonies. In addition, the residual broth (0.1 m) was cultured in RCM broth under an anaerobic condition at 37° C. for 48 hours.

Finally, the colony number of P. acnes on the plate was calculated; each plate should have 30˜300 colonies to be significant. Absorbance of the broth at 600 nm was measured to observe the growth changes of P. acnes.

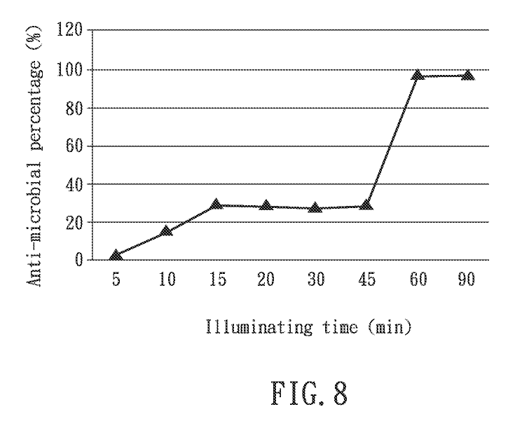

As shown in FIG. 8, after illumination with blue LED (Lux 5,090) and the agonist reagent for 60 minutes, the inhibition of P. acnes reaches 100%. This result demonstrates extremely significant inhibition.

Although the present invention has been explained in relation to its preferred embodiment, it is to be understood that many other possible modifications and variations can be made without departing from the spirit and scope of the invention as hereinafter claimed.

1-9. (canceled)

10. A photo-stimulation kit, comprising:
a photo-stimulation device which comprises a casing, a diffuser plate, a first illuminant module, and a controller, wherein the casing forms a deposition space and having a top surface and a lateral surface, and the top surface is provided with a light-output window; the diffuser plate covering the light-output window of the casing; the first illuminant module deposed in the deposition space of the casing and having a first light-emitting diode (LED) located under the diffuser plate, and the first light-emitting diode selected from a group consisting of a red LED, a yellow LED, a green LED, and a blue. LED, wherein the light emitted from the yellow LED and passing through the diffuser plate has an illuminance ranging from 1,000 to 3,500 lux (lx), the light emitted from the red LED passing through the diffuser plate has an illuminance ranging from 6,000 to 9,500 lux, the light emitted from the green LED passing through the diffuser plate has an illuminance ranging from 1,000 to 5,000 lux, and the light emitted from the blue LED passing through the diffuser plate has an illuminance ranging from 3,000 to 7,000 lux; and the controller module electrically connected with the first illuminant module and a power module;
a light-transmission plate covering a light-output hole provided in the lateral surface of the casing;
a second illuminant module deposed corresponding to the light transmission plate and emitting light which passes through the light transmission plate; and
an agonist agent which contains 0.5% to 2% calcium ion.

11. The photo-stimulation kit as claimed in claim 10, wherein the power module is an external power supply or is deposed in the deposition space of the casing.

12. The photo-stimulation kit as claimed in claim 11, wherein the controller module further comprises a charge socket, providing an electrical connection between the power module and the controller module.

13. The photo-stimulation kit as claimed in claim 10, wherein the controller module further comprises a power switch mounted on the surface of the casing to control power output of the power module.

14-15. (canceled)

16. The photo-stimulation kit as claimed in claim 10, wherein the controller module further comprises a mode switch mounted on the surface of the casing to turn on the first illuminant module or the second illuminant module.
